(19) United States
(12) Patent Application Publication
(10) Pub. No.: US 2017/0100383 A1
(43) Pub. Date: Apr. 13, 2017

(54) TOPICAL FORMULATIONS FOR PREVENTING SKIN INFECTION

(71) Applicant: Practical Solution, Inc., Sarasota, FL (US)

(72) Inventors: Zhou WAN, Sugar Land, TX (US); Steve GLASSEY, Nampa, ID (US); Jay MURPHY, Bradenton, FL (US); Wei XU, Sugar Land, TX (US)

(73) Assignee: Practical Solution, Inc., Sarasota, FL (US)

(21) Appl. No.: 15/291,214

(22) Filed: Oct. 12, 2016

Related U.S. Application Data
(60) Provisional application No. 62/241,048, filed on Oct. 13, 2015.

(51) Int. Cl. A61K 31/4425; A61K 31/155

(52) U.S. Cl. CPC A61K 31/4425; A61K 31/155

(57) ABSTRACT

A topical formulation comprising a first active agent that is pharmaceutically acceptable salt of a cationic bisbiguanide such as chlorhexidine digluconate (CHG), a secnd active agent which is a cationic quaternary ammonium compound such as cetylpyridinium chloride (CPC), methylisothiazolone, or chloroxylenol, and a propellant; and an aerosol dispenser containing the topical formulation. The topical formulation can be sprayed onto exposed skin to prevent or reduce the risk of skin infection.

### RELATED APPLICATIONS

This Application claims the benefit under 35 U.S.C. §119(e) of U.S. Provisional Application No. 62/241,048, filed on Oct. 13, 2015, entitled “TOPICAL FORMULATIONS FOR PREVENTING SKIN INFECTION”, the entire contents of which are incorporated by reference herein.

### BACKGROUND OF THE INVENTION

Skin infection is one of the top adverse events in wrestling. According to reports by the National Collegiate Athletic Association's (NCAA) Injury Surveillance System, ten percent of all time-loss injuries in wrestling are due to skin infections.

Skin infections can be caused by bacteria, viruses, and fungi. Many infections are contagious and may have serious effects on wrestler practices and competitions. Though wrestling teams frequently clean equipment and mats, skin infections are difficult to prevent since transmission of the pathogens is primarily through skin-to-skin contact. Adams, West J Med. 2001 May; 174(5): 352-353. Once skin is contaminated with microorganisms, they become colonized and eventually cause infection. Therefore, skin infection becomes a growing concern and its prevention is a priority for wrestlers and coaches.

Even though efforts have been made to improve wrestler hygiene practices, skin infection is still a top problem. Tinea infections, more commonly known as ringworm, are the most common skin infection transmitted through wrestling. en.wikipedia.org/wiki/Skin_infections_and_wrestling. Studies showed that the frequency of tinea corporis infection in wrestlers was found to range from 24% to 77%. Adams, West J Med. 2001 May; 174(5): 352-353. Bacterial infections caused by staphylococcal and streptococcal are also common among wrestlers. In addition, MRSA infections are likely to occur among wrestlers. Mayoclinic.org/diseases-conditions/mrsa/in-depth/mrsa/art-20047876. MRSA is a type of staphylococcal strains that are resistant to many common antibiotics. This ability makes MRSA infections much more difficult to cure.

Thus, it is desired to develop new products for preventing or reducing the risk of skin infections in sports, such as wrestling.

### SUMMARY OF THE INVENTION

In one aspect, the present disclosure provides a topical anti-infection formulation, comprising a first active agent, a second active agent, and a propellant. The first active agent is a pharmaceutically acceptable salt (e.g., dihydrochloride, diacetate, or digluconate) of a cationic bisbiguanide (e.g., chlorhexidine); and the second active agent is a cationic quaternary ammonium compound (e.g., cetylpyridinium chloride (CPC), benzalkonium chloride, benzethonium chloride, cetrimonium, cetrimide, dofanium chloride, and tetraethylammonium bromide), methylisothiazolone, or chloroxylenol. The propellant may be butane, isobutene, propane, and nitrogen gas.

In some examples, the first active agent is chlorhexidine digluconate (CHG) at a concentration of 0.0001-4% (w/v) (e.g., 0.1-1%), and the second active agent is CPC at a concentration of 0.01-1% (w/v), e.g., 0.05 -1% (w/v). In one example, the topical formulation comprises 0.5% (w/v) CHG and/or 0.75% (w/v) CPC. In another example, the topical formulation comprises 0.5% (w/v) CHG and/or 0.05% (w/v) CPC.

Any of the topical formulations described herein may further comprise (i) one or more of menthol, eucalyptol, thymol, a-Terpineol, camphor, lemon, tea tree , manuka, clove, cinnamon, thyme white, and lemongrass, and/or (ii) dimethicone or cyclomethicones. In some embodiments, the topical formulation further comprises menthol, eucalyptol, and dimethicone, e.g., at concentrations of 0.1-2% (w/v), 0.08-1% (v/v), and/or 0.2-0.5% (w/v), respectively. In one example, the topical formulation further comprises 1.0% (w/v) menthol, 0.5% (v/v) eucalyptol, and 0.3% (w/v) dimethicone.

Alternative or in addition, any of the topical formulations described herein may further comprise one or more of alcohol, propylene glycol, glycerin, and tocopheryl acetate. For example, the topical formulation may further comprise alcohol, propylene glycol, glycerin, and tocopheryl acetate. In one example, the formulation comprises 20-30% (v/v) alcohol, 3-10% (v/v) propylene glycol, 2-5% (v/v) glycerin, and 0.1-0.5% (w/v) tocopheryl actate.

In a specific example, the topical formulation as described herein comprises 0.5% (w/v) CHG, 0.75% (w/v) CPC, 1.0% (w/v) menthol, 0.5% (v/v) eucalyptol, 0.3% (w/v) dimenthicone, and nitrogen gas. In another specific example, the topical formulation as described herein comprises 0.5% (w/v) CHG, 0.05% (w/v) CPC, 0.5% (w/v) menthol, 0.08% (v/v) eucalyptol, 0.2% (w/v) dimenthicone, and nitrogen gas. Optionally, the topical formulation may further comprise alcohol, propylene glycol, glycerin, and tocopheryl acetate.

In another aspect, the present disclosure provides an aerosol dispenser, comprising any of the topical formulations described herein. In the aerosol dispenser, at least part of the propellant may be dissolved or emulsified in the solution comprising the other components of the topical formulation. Alternatively, whole or part of the propellant may be separate from the other components of the topical formulation (e.g., in different phases) prior to use.

Further, the present disclosure provides a method for preventing or reducing the risk of skin infection, the method comprising spraying any of the topical formulations described herein on exposed skin of a subject in need thereof. The subject may be a human subject. In some examples, the subject may be an athlete or a human who is participating in sports (e.g., wrestling) The topical formulation may be sprayed on the exposed skin prior to skin-to-skin or skin-to-surface contact. In some embodiments, the topical formulation is sprayed on the exposed skin no more than 3 to 4 times daily. Any of the methods may further comprise discontinuing application of the topical formulation to a subject if an allergic reaction occurs on the exposed skin of the subject where the topical formulation is sprayed.

Also within the scope of the present disclosure is a topical formulation as described herein for use in preventing or reducing the risk of skin infection, wherein the topical formulation is sprayed on an exposed skin area of a subject in need thereof, or use of the formulation for manufacturing a medicament for use in preventing or reducing the risk of skin infection.

The details of one or more embodiments of the invention are set forth in the description below. Other features or advantages of the present invention will be apparent from the following detailed description of several embodiments, and also from the appended claims.

### DETAILED DESCRIPTION OF THE INVENTION

Being subjected to extensive skin-to-skin contact, people engaged in sports, such as wrestlers, are examined carefully before each practice and competition. Besides clean and shower afterwards, athletes usually apply disinfectants immediately before activities to reduce skin bacterial levels. However, there are not many products serving this purpose on the market. Also, currently available products often have many drawbacks. For example, the available products are mostly liquid solution or cream, which makes it laborious to apply. Further, for area like the back, one would require someone else to help to put on and it is difficult and time consuming to spread it evenly. Moreover, the sensation of the current product are mostly either slippery or gluey after applied to the skin. With that said, users often feel that these products are troublesome to use and they tends to skip this important prepare procedure.

The present disclosure provides topical formulations, which can be placed in an aerosol dispenser, for preventing or reducing the risk of skin infection of subjects in need thereof, including human subjects who are or will be participating in sport events that would involve extensive skin contacts, for example, wrestling. The topical formulations described herein can be applied to exposed skin areas by a hands-free approach such as spraying using an aerosol spray dispensing system. Using an aerosol spray dispensing system, the light mist spray produced therefrom can cover hard-to-reach areas easily and dry quickly. The topical formulations described here are suitable for disinfecting skin areas prior to excise and competition, have good pharmacokinetic properties, including a rapid onset of action and long lasting activity against pathogens with relatively low side effects. It also has been demonstrated that the topical formulation is non-irritable and safe.

### Anti-Infection Topical Formulations

The topical formulation contains a mixture of selective antiseptics, disinfectants, and other optional components such as natural essential oils and their active components. More specifically, the topical formulation described herein comprises two active agents, one being a pharmaceutical acceptable salt of a cationic bisgiguanide and the other being a cationic quaternary ammonium compound, methylisothiazolone, or chloroxylenol, and optionally other components described herein.

Cationic bisgiguanide is a class of compounds having the generic formula of R¹ R² N.C(:NR⁶)NH.C(:NH)NH.CH₂ X—(CH₂)₃ NH.C(:NH)NH.C(:NR⁷)NR³ R⁴ V or a tautomer thereof. See U.S. Pat. No. 4,670,592. Such compounds include alexidine and chlorhexidine. TrialMichael et al., Crit Care Med. 2009; 37(6):1858-1865; and Wensen et al., J Thorac Dis. 2013 August; 5(4): 518-524. Examples of the pharmaceutically acceptable salts of the compounds for use in the topical formulations described herein include, but are not limited to, dihydrochloride, diacetate, and digluconate. In one specific example, the pharmaceutically acceptable salt of the bisgiguanide for use in the topical formulations is chlorhexidine digluconate (CHG). CHG, as well as other cationic bisgiguanide compounds, can exert immediate bactericide effects as well as cumulative effects that persist for hours and even longer after application, which improves efficacy after multiple application. Benson, et al. Infection control and Hospital Epidemiology (1990): 67-70; and Sogawa et al., Journal of Healthcare-associated Infection 2 (2010): 32-36. This character of longer activity provides protection several hours beyond initial application. These characteristics are ideal for serving the purpose of the topical formulations described herein, since wrestling or other sports excise or competitions may last for hours, and users can multi-apply the topical formulations during their activities and still have the protection.

In some examples, the concentration of the pharmaceutical salt of a cationic bisgiguanide, e.g., CHG, in the topical formulation may range from 0.0001-4% (w/v), e.g., 0.001-4% (w/v), 0.01-4% (w/v), 0.1-3% (w/v), 0.1-2% (w/v), or 0.1-1% (w/v). In one example, the concentration of CHG is 0.5% (w/v). Using such a low concentration of CHG can reduce potential skin allergy reactions while still preserving the anti-infection activity. Calogiuri et al., Chlorhexidine Hypersensitivity: A Critical and Updated Review. J Allergy Ther 2013, 4:4; and Nagendran et al., Occup Med (Loud). 2009 June; 59(4):270-2.

Since CHG is a pharmaceutically cationic compound, it is compatible with other cationic and non-ionic substances. Quirynen et al., J Clin Periodontol. 2005 April; 32(4):390-400; Pons et al., J Appl Bacteriol. 1992 November; 73(5):395-400; U.S. Pat. No. 5,756,145; and WO 97/25085. The topical formulations described herein further comprise a second active agent, which can be a cationic quaternary ammonium compound such as cetylpyridinium chloride (CPC), methylisothiazolone, or chloroxylenol. The use of this second active agent can solve potential efficacy and susceptibility problems in association with chlorhexidine (Homer et al., J Antimicrob Chemother. 2012 November; 67(11):2547-59), expand the anti-infection activity of the topical formulation to additional types of pathogens, particularly drug-resistant pathogens, and/or exhibit synergistic effect with the salt of cationic bisgiguanide. Thus, the co-use of the two active agents described herein enhances the antimicrobial activity of the topical formulation.

Cationic quaternary ammonium compounds are positively charged polyatomic ions of the structure NR₄⁺, in which R can be an alkyl group or an aryl group, or a salt thereof. Examples of cationic quaternary ammonium compounds for use in the instant disclosure include, but are not limited to, cetylpyridinium chloride (CPC), benzalkonium chloride, benzethonium chloride, cetrimonium, cetrimide, dofanium chloride, and tetraethylammonium bromide. Other examples include methylbenzethonium chloride, cetalkonium chloride, cetrimonium, cetrimide, and domiphen bromide.

The topical formulation described herein may optionally further comprise one or more of the following components: natural essential oils, camphor, lemon, tea tree , manuka, clove, cinnamon, thyme white, lemongrass, alcohol, and their active components, thymol, menthol, and/or eucalyptol. Essential oils have a broad spectrum of antimicrobial activity and this property has been proved in therapeutics, including skin cleansing (MRSA decolonisation) and treatment of necrotic ulcers. Sivananthan et al., Diabetes Research and Clinical Practice. 2003; 62(1):65-66; Warnke et al., J Craniomaxillofac Surg. 2009; 37(7):392-397; and Warnke et al., Phytomedicine, 2006; 13(7):463-467. For example, essential oils, such as tea tree oil, eucalyptus oil, thymol, and their active ingredients may improve skin antisepsis when combined with chlorhexidine digluconate. Hendry et al., Int J Mol Sci. 2012 Oct. 30; 13(11):14016-25; Hendry et al., J Antimicrob Chemother. 2009 December; 64(6):1219-25; Karpanen et al., J Antimicrob Chemother. 2008 November; 62(5):1031-6; and Filoche et al., Oral Microbiol Immunol. 2005 August; 20(4):221-5.

In some embodiments, it may further comprise dimethicone or cyclomethicones. Alternatively or in addition, the topical formulation may further comprise one or more of alcohol, propylene glycol, glycerin, and tocopheryl acetate.

### Aerosol Dispenser

As used herein, “aerosol” refers to products which are dispensed as a mist, stream, spray, powder or even a foam. Aerosol dispensers are an exemplary vehicle for the storage of and delivery of the topical formulations described herein. The aerosol dispensers described herein include a container, such as a pressurizable canister, which contains the topical formulation described herein to be dispensed. A pressurised propellant is also provided to the aerosol dispenser and is used to provide a force sufficient to discharge the topical formulation from the container. The user actuates the aerosol dispenser by for example pressing an actuator button in order to dispense the topical formulation from the aerosol dispenser.

The aerosol dispenser contains a propellant such that the topical formulation contained therein can be applied by aerosol spray. A propellant is a chemical substance used in the production of energy or pressurized gas that is subsequently used to create movement of a fluid or to generate propulsion of a vehicle, projectile, or other object. The propellant for use in making aerosol spray cans may be a pressurized gas, e.g., nitrogen gas, nitrous oxide, carbon dioxide, in equilibrium with the liquid. Other examples of propellant include, but are not limited to, volatile hydrocarbons, (e.g., propane, n-butane and isobutane), dimethyl ether (DME), and methyl ethyl ether. The propellant in the aerosol dispenser may be in an amount from about 3 to about 20 weight percent of the total composition for pressurized discharge of the topical formulation.

When the aerosol dispenser described herein uses a liquefied gas-type propellant, the container can be loaded with the formulation containing the propellant to a pressure approximately equal to, or to a pressure slightly greater than, the vapor pressure of the propellant. The propellant may be mixed with the formulation before loading (e.g., be a component of the topical formulation). Alternatively, it may be loaded into the dispenser together with the formulation (e.g., concurrently or in sequential). Since the container is pressurized to approximately the vapor pressure of the propellant, some of the propellant may be dissolved or emulsified in the formulation. The remainder of the propellant can be in the vapor phase and fills the head space of the aerosol dispenser. As the product is dispensed, the pressure in the container remains approximately constant as liquid propellant evaporates to replenish discharged vapor. A liquefied gas propellant keeps the pressure approximately constant in the aerosol dispenser until the contents are exhausted, thus ensuring a generally consistent spray performance throughout the lifetime of the can.

The aerosol dispenser described herein may be prepared by mixing all the components except the propellant and adding such mixture to a suitable container to be pressurized, after which the propellant is added under suitable pressurized conditions by a conventional method. See, e.g., U.S. Pat. No. 5,906,808. For example, in the case of dimethyl ether, a supply of the propellant in liquid form at a pressure of 500 to 800 psi may be added to a container of the other mixed components to produce a pressurized container holding a homogeneous mixture of propellant and the other components, with an internal pressure of about 60 psi in the container.

The aerosol dispenser may not be stored below a freezing temperature or above 120° F.

### Use of Topical Formulation for Preventing Skin Infection

The topical formulation may be applied to an exposed skin area of a subject in need of the treatment by aerosol spray from the aerosol dispenser to prevent or reduce the risk of skin infection. For example, the formulation may be sprayed on exposed skin (e.g., cover all areas that are likely to have contact) and let dry prior to skin-to-skin contact or skin-to-surface contact. One may shake well the contents in the aerosol dispenser described herein prior to use and may hold the aerosol dispenser described herein at least 12 inches away from the skin and spray in a pattern that will cover desired areas. Eye contact or inhaling the contents of the aerosol dispenser should be avoided as it could be harmful. The formulation may be applied to desired skin areas no more than 3 to 4 times every day. If needed, the skin area where the formulation is applied may be washed with soap and water before bed or when no further contact is likely. The use of large quantities of the formulation may be avoided. If rash or other allergic reaction occurs, one may discontinue use and, if necessary, consult a physician or health care expert.

The topical formulation described herein may be applied to any subject (e.g., a human subject) having, suspected of, or at risk for skin infection. In some instances, the human subject can be an adult or a child of 8 years or older. Such a subject may be a human subject involved in sports activities that are likely to have skin-to-skin contact or skin-to-surface contact, for example, wrestling. The topical formulation may be sprayed on expose skin areas of such a human subject right before his or her participation in the sports activities. If necessary, the topical formulation may be applied to the subject one or more times again during the sport activity as well.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLE 1

### Minimum Inhibitory Concentration (MIC) Assay Showing Synergistic Anti-Microbial Effect of CHG and CPC

A minimum inhibitory concentration (MIC) assay was carried to investigate the lowest concentration of CHG, CPC, and a combination thereof, that inhibits the visible growth of Staphyloccocus aureus, Bacillus subtilis and Candida albicans after 48-hour incubation using broth microdilution.

Briefly, CHG, CPC, or a combination thereof was dissolved in distill water at different concentrations as indicated below (initial concentrations):

CHG: 5 g/L

CPC: 0.5 g/L

CHG+CPC: 5 g/L (CHG) and 0.5 g/L (CPC)

CHG, CPC, and CHG+CPC having the above-noted concentrations were diluted to fold serial dilutions with distill water. 2.5 ml of CHG, CPC, or CHG+CPC were mixed with 2.5 ml of broth comprising growth media, such as Mueller-Hinton Broth (MHB), to obtain 10-, 100-, 1,000-, 2,000-, 4,000-, 8,000-, 16,000-, or 32,000-concentration dilution. Staphyloccocus aureus strain ATCC 6538, Bacillus subtilis ATCC9372 or Candida albicans ATCC10231 each (0.1 ml of a bacterial suspension having 10⁸ cfu/ml bacteria) were inoculated into the mixture. A broth inoculated with the same microorganisms and having no CHG and CPC was used as a positive control and a broth having CHG and/or CPC but no microorganisms was used as a blank control. Test tubes containing the mixtures were placed in a 37° C. incubator and incubated for 48 hours. The concentration of bacterial cells in each test tube was measured. The inhibitory rates were calculated as below:

[(Positive control average colony count−Test average colony count)/Positive control average colony count]×100%

Positive controls are the tubes having broth and microorganisms but no antimicrobial agents.

The results obtained from this study are provided in Tables 1-3 below:

TABLE 1
Anti-microbial Activities of CHG, CPC, and a Combination
Thereof (%) against Staphylococcus aureus ATCC6538

| | Antimicrobial agent | | |
|---|---|---|---|
| Dilutions | CHG | CPC | CHG + CPC |
| 1* | 100 | 100 | 100 |
| 1/10 | 100 | 100 | 100 |
| 1/100 | 100 | 100 | 100 |
| 1/1000 | 100 | 100 | 100 |
| 1/2000 | 100 | 98.49 | 100 |
| 1/4000 | 100 | 36.27 | 100 |
| 1/8000 | 78.43 | 24.51 | 99.32 |

The synergistic effect of CHG and CPC was observed at 1/8000 dilution.

TABLE 2
Anti-microbial Activities of CHG, CPC, and a Combination
Thereof (%) against Bacillus subtilis ATCC9372

| | Antimicrobial agent | | |
|---|---|---|---|
| Dilutions | CHG | CPC | CHG + CPC |
| 1* | 100 | 100 | 100 |
| 1/10 | 100 | 100 | 100 |
| 1/100 | 100 | 100 | 100 |
| 1/1000 | 100 | 97.73 | 100 |
| 1/2000 | 100 | 96.45 | 100 |
| 1/4000 | 100 | 96.25 | 100 |
| 1/8000 | 99.99 | 93.00 | 100 |
| 1/16000 | 99.50 | 90.05 | 99.63 |
| 1/32000 | 90.83 | 62.50 | 95.00 |

The synergistic effect of CHG and CPC was observed at 1/32000 dilution

TABLE 3
Anti-microbial Activities of CHG, CPC, and a Combination
Thereof (%) against Candida albicans ATCC10231

| | Antimicrobial agent | | |
|---|---|---|---|
| Dilutions | CHG | CPC | CHG + CPC |
| 1* | 100 | 100 | 100 |
| 1/10 | 100 | 100 | 100 |
| 1/100 | 100 | 99.90 | 100 |
| 1/1000 | 97.76 | 91.36 | 98.61 |
| 1/2000 | 93.92 | 89.36 | 95.25 |
| 1/4000 | 90.41 | 45.90 | 93.29 |

The synergistic effect of CHG and CPC was observed at 1/4000 dilution.

### EXAMPLE 2

### Exemplary Formulations Comprising CHG and CPC

Provided in Tables 4-7 below are components of a number of exemplary aerosol formulations as described herein:

TABLE 4
Components of Formula I

| Ingredients | Percentage % | |
|---|---|---|
| Water/propellant | | |
| Alcohol | 20-30 | (v/v) |
| Chlorhexidine digluconate (CHG) | 0.1-1 | (w/v) |
| Cetylpyridinium chloride (CPC) | 0.05-0.1 | (w/v) |
| Menthol | 0.1-2 | (w/v) |
| Eucalyptol | 0.08-1 | (v/v) |
| Dimethicone | 0.2-0.5 | (w/v) |
| Propylene glycol | 3-10 | (v/v) |
| Glycerin | 2-5 | (v/v) |
| Tocopheryl Acetate | 0.1-0.5 | (w/v) |

TABLE 5
Components of Formula II

| | Percentage | 1 Gallon | | | |
|---|---|---|---|---|---|
| Ingredients | % | (3785 ml) | | 55 Gallon | |
| Alcohol | 23.5 | 889 | ml | 49 | L |
| Chlorhexidine digluconate (CHG) | 0.5 | 18.9 | g | 1040 | g |
| Cetylpyridinium chloride (CPC) | 0.05 | 1.9 | g | 104 | g |
| Menthol | 0.5 | 18.9 | g | 1040 | g |
| Eucalyptol | 0.08 | 3 | ml | 167 | ml |
| Cyclomethicone | 2 | 76 | ml | 4.16 | L |
| Propylene glycol | 3 | 114 | ml | 6.25 | L |
| Tocopheryl Acetate | 0.2 | 7.6 | g | 416 | g |
| Propellant (isobutane) | 60-70 | | | | |
| Water | | | | | |

TABLE 6
Components of Formula III

| Ingredients | Percentage % | |
|---|---|---|
| Alcohol | | |
| Chlorhexidine digluconate (CHG) | 0.5 | (w/v) |
| Cetylpyridinium chloride (CPC) | 0.75 | (w/v) |
| Menthol | 1.0 | (w/v) |
| Eucalyptol | 0.5 | (v/v) |
| Dimethicone | 0.3 | (w/v) |
| Propylene glycol | 3 | (v/v) |
| Tocopheryl Acetate | 0.3 | (w/v) |
| Glycerin | 2 | (v/v) |
| Nitrogen Gas | | |
| Water | | |

TABLE 7
Components of Formula IV

| Ingredients | Percentage % | |
|---|---|---|
| Alcohol | 23.5% | |
| Chlorhexidine digluconate (CHG) | 0.5 | (w/v) |
| Cetylpyridinium chloride (CPC) | 0.05 | (w/v) |
| Menthol | 0.5 | (w/v) |
| Eucalyptol | 0.08 | (v/v) |
| Dimethicone | 0.2 | (w/v) |

| | |
|---|---|
| Propylene glycol | 3 |
| Tocopheryl Acetate | 0.2 |
| Glycerin | 2 |
| Nitrogen Gas | |
| Water | |

The formulas listed above, e.g., Formula III or Formula IV, can be used via aerosol spray to treat ringworm, impetigo and staph infection and the results can be better than conventional therapies.

### EXAMPLE 3

### Antimicrobial Efficacy Test on Animal Skin

The anti-microbial effect of the composition described herein were tested on six healthy rabbits, which have no skin disorders, in this study using modified Cup Scrub test.

Each rabbit was treated to produce several test sites and control sites on its skin. Each test site and control site was delineated by a sterile glass cylinder (3 cm diameter). The test solution (Formula IV) was spread on a test site and a control solution was spread on a control site. Each rabbit has at least one test site and at least one control site.

10 μl of a bacterial solution, containing Staphylococcus aureus ATCC6538 at 10⁶ to 10⁷ cfu, were applied to all test sites and control sites within the area of sampling cylinders. All tested sites and control sites were then covered with sterile plastic cups for 2 hours. Afterwards, stainless steel cylinders were placed over the test and control sites. A protocol-specified volume of Sterile Stripping Fluid is dispensed into the cylinders, and a sterile scrubbing tool was used to harvest surviving bacteria from each test site and each control site.

The sampling fluids from each site were pooled and mixed thoroughly. Ten-fold serial dilutions of each sample were prepared in the dilution fluid. Samples with suitable dilutions were plated on blood agar (TSA w/5% Sheep Blood) and the plates were incubated for 35° C±2° C. for 48 h±4 h. The number of colonies on each plate was counted.

The inhibition efficiency was calculated following the formula below:

% inhibition=(Mean colony counts of control plates−Mean colony counts of test plates)×100/Mean colony counts of control plates

As shown in Table 8 below, the formulation described herein successfully inhibited bacterial growth for at least two hours.

TABLE 8
Effectiveness of tested Formulation
in inhibiting S. aureus growth (%)

| Test Animals | Reduction Rate | Mean Reduction Rate |
|---|---|---|
| 1 | 100 | 100 |
| 2 | 100 | |
| 3 | 100 | |
| 4 | 100 | |
| 5 | 100 | |
| 6 | 100 | |

### EXAMPLE 4

### Skin Irritation Test

The topical formulation was tested in this study to examine whether it would cause irritation on skin as follows.

Four rabbits were shaved to expose a skin a skin surface. The test formulation (Formula IV) was applied thereon. The test sites were then covered with a gauze patch for four hours. After the gauze patch was removed, degrees of irritation at the tested sites were examined at 1, 2, 48, and 72 hours intervals. Shaved skin surfaces not treated by the test formulation were used as a control.

As shown in Table 9 below, the test formulation did not cause skin irritation (e.g., redness or edema), indicating that it is not an irritant.

TABLE 9
Skin Irritation Test of Formula IV

| | | | 1 h | | | | | | 24 h | | | | | | 48 h | | | | | | 72 h | | | | | |
|---|---|---|---|---|---|---|---|---|---|---|---|---|---|---|---|---|---|---|---|---|---|---|---|---|---|---|
| | | Weight | Test | | | Control | | | Test | | | Control | | | Test | | | Control | | | Test | | | Control | | |
| Animal | | kg | R | E | S | R | E | S | R | E | S | R | E | S | R | E | S | R | E | S | R | E | S | R | E | S |
| 1 | ♂ | 2.2 | 0 | 0 | 0 | 0 | 0 | 0 | 0 | 0 | 0 | 0 | 0 | 0 | 0 | 0 | 0 | 0 | 0 | 0 | 0 | 0 | 0 | 0 | 0 | 0 |
| 2 | ♂ | 2.2 | 0 | 0 | 0 | 0 | 0 | 0 | 0 | 0 | 0 | 0 | 0 | 0 | 0 | 0 | 0 | 0 | 0 | 0 | 0 | 0 | 0 | 0 | 0 | 0 |
| 3 | ♀ | 2.0 | 0 | 0 | 0 | 0 | 0 | 0 | 0 | 0 | 0 | 0 | 0 | 0 | 0 | 0 | 0 | 0 | 0 | 0 | 0 | 0 | 0 | 0 | 0 | 0 |
| 4 | ♀ | 2.0 | 0 | 0 | 0 | 0 | 0 | 0 | 0 | 0 | 0 | 0 | 0 | 0 | 0 | 0 | 0 | 0 | 0 | 0 | 0 | 0 | 0 | 0 | 0 | 0 |

R: Redness;
E: Edema;
S: Score

### EXAMPLE 5

### Acute Systemic Toxicity Test of Topical Formulation

The acute systemic toxicity of the topical formulation described herein was examined as follows, using Formula IV as an example.

A single dose (5 g/kg) of Formula IV was administered to 20 mice orally and occurrence of adverse effects was observed in 14 days after the administration. As shown in Table 10, no mouse was dead after taking the formulation.

TABLE 10
Death Rate of Mice in Fourteen Days After
Oral Administration of Formula IV

| Dosage (g/kg) | Mice Number (Total) | Death of Mice | Death Rate % |
|---|---|---|---|
| 5.0 | 20 | 0 | 0 |

The tested mice were also dissected 14 days after the administration to examine whether organ abnormality occurs. The results showed that no abnormality occurs in major organs, including heart, liver, lung, intestine, and kidney. The LD₅₀ value determined in this study is greater than 5000 mg/kg.

Taken together, this study shows that the topical formulation described herein is non-toxic.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A topical formulation, comprising a first active agent, a second active agent, and a propellant, wherein:
(i) the first active agent is a pharmaceutically acceptable salt of a cationic bisbiguanide; and
(ii) the second active agent is selected from the group consisting of a cationic quaternary ammonium compound, methylisothiazolone, and chloroxylenol.

2. The topical formulation of claim 1, wherein the bisbiguanide is chlorhexidine.

3. The topical formulation of claim 1, wherein the pharmaceutically acceptable salt is dihydrochloride, diacetate, or digluconate.

4. The topical formulation of claim 1, wherein the cationic quaternary ammonium compound is cetylpyridinium chloride (CPC), benzalkonium chloride, benzethonium chloride, cetrimonium, cetrimide, dofanium chloride, or tetraethylammonium bromide.

5. The topical formulation of claim 1, wherein the first active agent is chlorhexidine digluconate (CHG) at a concentration of 0.0001-4% (w/v), and the second active agent is CPC at a concentration of 0.01-1% (w/v).

6. The topical formulation of claim 5, wherein the concentration of CHG is 0.1-1% (w/v).

7. The topical formulation of claim 5, wherein the concentration of CPC is 0.05 -1% (w/v).

8. The topical formulation of claim 1, further comprising (i) one or more of menthol, eucalyptol, thymol, a-Terpineol, camphor, lemon, tea tree, manuka, clove, cinnamon, thyme white, and lemongrass, and/or (ii) dimethicone or cyclomethicones.

9. The topical formulation of claim 1, further comprising menthol, eucalyptol, and dimethicone.

10. The topical formulation of claim 9, wherein the formulation comprises 0.1-2%(w/v) menthol, 0.08-1% (v/v) eucalyptol, and/or 0.2-0.5% (w/v) dimethicone.

11. The topical formulation of claim 1, wherein the formulation comprises 0.5% (w/v) CHG.

12. The topical formulation of claim 1, wherein the formulation comprises 0.75% (w/v) CPC.

13. The topical formulation of claim 1, wherein the formulation comprises 1.0% (w/v) menthol, 0.5% (v/v) eucalyptol, and 0.3% (w/v) dimethicone.

14. The topical formulation of claim 1, wherein the formulation comprises 0.5% (w/v) menthol, 0.08% (v/v) eucalyptol, and 0.2% (w/v) dimethicone.

15. The topical formulation of claim 1, wherein the formulation further comprises one or more of alcohol, propylene glycol, glycerin, and tocopheryl acetate.

16. The topical formulation of claim 15, wherein the formulation further comprises alcohol, propylene glycol, glycerin, and tocopheryl acetate.

17. The topical formulation of claim 16, wherein the formulation comprises 20-30% (v/v) alcohol, 3-10% (v/v) propylene glycol, 2-5% (v/v) glycerin, and 0.1-0.5% (w/v) tocopheryl actate.

18. The topical formulation of claim 1, wherein the propellant is selected from the group consisting of butane, isobutene, propane, and nitrogen gas.

19. The topical formulation of claim 1, wherein the topical formulation comprises 0.5% (w/v) CHG, 0.75% (w/v) CPC, 1.0% (w/v) menthol, 0.5% (v/v) eucalyptol, 0.3% (w/v) dimenthicone, and nitrogen gas.

20. The topical formulation of claim 1, wherein the topical formulation comprises 0.5% (w/v) CHG, 0.05% (w/v) CPC, 0.5% (w/v) menthol, 0.08% (v/v) eucalyptol, 0.2% (w/v) dimenthicone, and nitrogen gas.

21. The topical formulation of claim 19, wherein the topical formulation further comprises alcohol, propylene glycol, glycerin, and tocopheryl acetate.

22. An aerosol dispenser, comprising a topical formulation of claim 1.

23. A method for preventing or reducing the risk of skin infection, comprising spraying a topical formulation of claim 1 on exposed skin of a subject in need thereof.

24. The method of claim 23, wherein the subject is a human subject.

25. The method of claim 23, wherein the human subject is an athlete.

26. The method of claim 23, wherein the topical formulation is sprayed on the exposed skin prior to skin-to-skin or skin-to-surface contact.

27. The method of claim 23, wherein the topical formulation is sprayed on the exposed skin no more than 3 to 4 times daily.

28. The method of claim 23, further comprising discontinuing application of the topical formulation to a subject if an allergic reaction occurs on the exposed skin of the subject where the topical formulation is sprayed.
